(19) United States
(12) Patent Application Publication
(10) Pub. No.: US 2013/0066100 A1
(43) Pub. Date: Mar. 14, 2013

(54) PROCESS FOR PREPARING CATALYST USED IN PRODUCTION OF UNSATURATED ALDEHYDE AND/OR UNSATURATED CARBOXYLIC ACID BY DEHYDRATION REACTION OF GLYCERIN, AND CATALYST OBTAINED

(71) Applicants: Yasuhiro Magatani, SanyoOnoda-shi (JP); Kimito Okumura, SanyoOnoda-shi (JP); Jean-Luc Dubois, Millery (FR)

(72) Inventors: Yasuhiro Magatani, SanyoOnoda-shi (JP); Kimito Okumura, SanyoOnoda-shi (JP); Jean-Luc Dubois, Millery (FR)

(73) Assignee: NIPPON KAYAKU KABUSHIKI KAISHA, Chiyoda-ku, Tokyo (JP)

(21) Appl. No.: 13/501,929

(22) PCT Filed: Oct. 15, 2010
(86) PCT No.: PCT/JP2010/068644

§ 371 Date: Nov. 29, 2012

(30) Foreign Application Priority Data
Oct. 15, 2009 (JP) 2009-238532

(51) Int. Cl. B01J 37/08; C07C 253/26; C07C 45/52; C07C 51/16; B01J 27/188; B01J 35/02

(52) U.S. Cl. 558/315; 502/100; 502/210; 568/486; 562/532

(57) ABSTRACT

A process for preparing a catalyst used in a production of acrolein and acrylic acid by dehydration reaction of glycerin, characterized by the steps of mixing a solution of heteropolyacid or constituents of heteropolyacid, a solution of at least one metal selected from elements belonging to Group 1 to Group 16 of the Periodic Table of Elements or its onium and a carrier to obtain a solid substance, and then of effecting at least one time of calcination before said solid substance is used in the dehydration reaction of glycerin.

A catalyst obtained by the process for use in a production of acrolein and acrylic acid by dehydration reaction of glycerin.

A process for preparing acrolein by catalytic dehydration of glycerin carried out in the presence of the catalyst and under a pressurized condition.

A process for preparing acrylic acid obtained by oxydation of acrolein obtained.

A process for preparing acrylonitrile obtained by ammoxidation of acrolein obtained.

### TECHNICAL FIELD

This invention relates to improvement in a process for preparing a catalyst used in dehydration reaction of glycerine to produce unsaturated aldehyde and/or unsaturated carboxylic acid.

This invention relates also to an improved catalyst used in the dehydration reaction of glycerine.

This invention relates further to a process for preparing unsaturated aldehyde and/or unsaturated carboxylic acid carried out in the presence of the dehydration catalyst.

### BACKGROUND ART

Glycerin is obtained in large amount as a byproduct when bio-fuel is produced from bio resources that do not depend on fossil resources, and research of new uses of glycerin is under development.

WO2007/058221 discloses a process for producing acrolein by dehydration reaction of glycerin in gas-phase in the presence of heteropolyacid used as a solid acid catalyst. The heteropolyacid is those of Group 6 element such as tungstosilicic acid, tungstophosphoric acid and phosphomolybdic acid. These heteropolyacids are supported on bi-modal pore size distribution silica carrier and produce acrolein at a yield of 86%. This dehydration reaction of glycerin, however, is effected without oxidation gas but using nitrogen stream as carrier gas, so that deposition of carbon increase seriously and hence there is a problem of deterioration in time of stability, activity and selectivity of the catalysis.

Tsukuda et al. “Production of acrolein from glycerol over silica-supported heteropolyacid” CATALYSIS COMMUNICATIONS, vol. 8, no. 9, 21 July 2007, pp 1349-1353, Chai et al., “Sustainable production of acrolein: gas phase dehydration of glycerol over 12-tungstophosphoric acid supported on ZrO₂ and SiO₂”, GREEN CHEMISTRY, vol. 10, 2008, pp. 1087-1093, and Chai et al., “Sustainable production of acrolein: preparation and characterization of zirconia-supported 12-tungstophosphoric acid catalyst for gas phase dehydration of glycerol”, APPLIED CATALYSIS A: GENERAL, vol. 353, 2009, pp. 213-222 disclose that silica or zirconia-supported heteropolyacid is effective as a catalyst for dehydration of glycerol. However, there is no usable catalyst in the industrial scale at higher performance.

WO2007/058221 (Nippon Shokubai) discloses a process for dehydrating polyhydric alcohols by using a catalyst containing an element of group 6 (Cr, Mo, W), in particular, comprising a heteropolyacid which can be supported on a carrier containing Al, Si, Ti or Zr. Examples show the acrolein yield of 70% for PW/Al₂O₃ 70% for PW/ZrO₂, 87% for SiW/SiO₂ but the conversion decreases from 100% to 70% in 8 hours.

U.S. patent No. 2009054538 (BATTELLE) discloses catalyst composition comprising phosphotungstic or phosphomolybdic acid on silica support and the acrolein yields obtained are not over 71% with the catalysts.

U.S. Pat. No. 5,919,725 discloses a catalyst comprising heteropoly salts and heteropolyacid salts deposited on a porous support of silica, zirconia and titania. This catalyst is used for aromatic alkylation such as alkylation of phenol with olefins but there is no mention of glycerol dehydration.

U.S. Pat. No. 4,983,565 discloses a process for preparing a catalyst composition by impregnating titania pellets with an aqueous solution consisting of tungstosilicic acid or molybdosilicic acid or their salts followed by drying and calcination. The catalyst composition is prepared preferably by impregnating a preformed pellet by immersing titania pellets in an aqueous solution of the tungstosilicic acid or molybdosilicic acid, for example. However, this patent teaches nothing about such a feature defined in the present invention that protons in the heteropolyacid are exchanged by at least one cation selected from elements belonging to Group 1 to Group 16 of the Periodic Table of Elements. Still more, this catalyst is used to prepare linear polyethylenepolyamine but there is no mention in dehydration of glycerol.

JP-2005-131470-A1 discloses a fine metal particle carrier used as a catalyst for oxidation-reduction reaction and acid-base reactions. This carrier comprises a tungsten-containing porous carrier on which fine metal particles containing the group II element are supported.

JP-2007-137785-A1 discloses a catalyst used in gas-phase dehydration reaction of glycerine. This catalyst contains at lest one of the group VI elements. JP-2007-268364-A1 discloses a supported catalyst used in dehydration reaction of glycerine, comprising a carrier on which P and alkali metal (M) are supported. The alkali metal is more than one of Na, K and Cs, a molar ratio (M/P) of the alkali metal to P being less than 2.0.

Inventors disclosed, in JP-2008-530150-A1 and JP-2008-530151-A1, a process for preparing acrolein by dehydration reaction of glycerine, effected in the presence of molecular oxygen and of strong acid solid having Hammett acidity Ho of −9 to −18.

Inventors have proposed also, in PCT/JP2009/057818, PCT/JP2009/057819 and other pending applications, an improved dehydration catalyst comprising mainly a compound in which protons in a heteropolyacid are exchanged at least partially with at least one cation selected from elements belonging to Group 1 to Group 16 of the Periodic Table of Elements.

### DISCLOSURE OF INVENTION

### Technical Problems

Inventors found an improved process for preparing a catalyst used in dehydration reaction of glycerin, which can improve the yield of products of unsaturated aldehyde and unsaturated carboxylic acid.

Inventors found also that the catalyst obtained by the improved process permits to carry out the dehydration reaction of glycerin under a pressurized condition for longer operation duration, so that the unsaturated aldehyde and unsaturated carboxylic acid can be produced at higher productivity and for longer running time.

Therefore, it is an object of the present invention to provide a process for producing unsaturated aldehyde and unsaturated carboxylic acid by a catalytic dehydration reaction of glycerin that can be operated for longer time duration under a pressurized condition.

Another object of this invention is to provide an improved catalyst obtained by the above process that can produce unsaturated aldehyde and unsaturated carboxylic acid at the high yield and at a higher productivity.

Still another object of this invention is to provide unsaturated aldehyde and unsaturated carboxylic acid by the catalytic dehydration reaction even under the pressurized operation condition at higher yield and at higher productivity.

### Technical Solution

From the first aspect, the present invention provides a process for preparing a catalyst used in a production of acrolein and acrylic acid by dehydration reaction of glycerin, characterized by the steps of mixing a solution of at least one metal selected from elements belonging to Group 1 to Group 16 of the Periodic Table of Elements or its onium with a solution of heteropolyacid or constituents of heteropolyacid, and of calcinating the resulting solid substance directly or after the resulting solid substance is supported on a carrier.

From second aspect, the present invention provides a process for preparing a catalyst used in a production of acrolein and acrylic acid by dehydration reaction of glycerin, characterized by the steps of either mixing a solution of heteropolyacid or constituents of heteropolyacid with a carrier, and then adding a solution of at least one metal selected from elements belonging to Group 1 to Group 16 of the Periodic Table of Elements or its onium to the resulting mixture, or mixing a solution of at least one metal selected from elements belonging to Group 1 to Group 16 of the Periodic Table of Elements or its onium with a carrier, and then adding a solution of heteropolyacid or constituents of heteropolyacid to the resulting mixture, and then calcinating the resulting solid substance to obtain the catalyst.

From the further aspect, the present invention provides a process for preparing a catalyst used in a production of acrolein and acrylic acid by dehydration reaction of glycerin, characterized by mixing a solution of heteropolyacid or constituents of heteropolyacid, a solution of at least one metal selected from elements belonging to Group 1 to Group 16 of the Periodic Table of Elements or its onium and a carrier to obtain a solid substance, and then effecting at least one time of calcination before said solid substance is used in the dehydration reaction of glycerin.

The above process inventions may have following features (1) and (2) taken separately or in combination:
(1) The calcination is carried out in air, in inert gas or in a mixture of oxygen and inert gas or under a reduced gas of hydrogen and inert gas.
(2) The calcination is effected at a temperature of 150° C. to 900° C. for 0.5 to 20 hours.

The present invention provides further a catalyst obtained by the above processes for production of acrolein and acrylic acid by dehydration reaction of glycerin.

The present invention provides further a process for preparing acrolein by catalytic dehydration of glycerin under a pressurized condition and carried out in the presence of the catalyst.

The present invention provides further a process for preparing acrylic acid comprising a first step of catalytic dehydration of glycerin under a pressurized condition and carried out in the presence of the catalyst, and a second step of gas phase oxidation of the gaseous reaction product containing acrolein formed by the dehydration reaction.

The above processes may have following features (1) to (7) taken separately or in combination:
(1) The dehydration of glycerin is effected in the presence of oxygen gas with the conditions disclosed for example in WO 06/087083 or WO 06/114506.
(2) The dehydration of glycerin is effected in the presence of a gas containing propylene, as disclosed for example in WO 07/090990 and WO 07/090991, that is say to carry out the glycerin dehydration stage beneath the propylene oxidation reactor of the conventional process, taking benefit of the high temperature of the gas coming out of that stage containing mainly acrolein and some remaining propylene.
(3) The dehydration of glycerin is carried out in a plate heat exchanger type reactor or in a fixed bed reactor or in a fluidized bed type reactor or in a circulating fluidized bed or in a moving bed.
(4) The process for preparing acrylic acid has an intermediate step of partial condensation and removal of water and heavy by-products issuing from the dehydration step, as described for example in WO 08/087315.
(5) The catalytic dehydration of glycerin is effected under a pressurized condition of relative pressure of 0.01 MPa to 1 MPa.
(6) The resulting acrolein from the catalytic dehydration of glycerin is further oxidized to produce acrylic acid, according to the methods well known to the skilled in the arts.
(7) The process for preparing acrylic acid further comprises the steps of collecting the resultant acrylic acid as a solution by using water or a solvent and then of purifying the resultant solution containing acrylic acid by using for example distillation and/or crystallization.

The present invention provides further a process for preparing acrylonitrile, characterized in that acrolein obtained by the above process for preparing acrolein by catalytic dehydration of glycerin is subjected to ammoxidation, as described for example in WO 08/113927.

### Advantageous Effect

By using the improved catalyst, products of unsaturated aldehyde and unsaturated carboxylic acid by the dehydration reaction of glycerin can be produced at higher yield.

By using the improved catalyst, the dehydration reaction of glycerin can be carried out even under a pressurized condition for longer operation duration, so that the unsaturated aldehyde and unsaturated carboxylic acid can be produced at higher productivity and for longer running time.

### BEST MODE FOR CARRYING OUT THE INVENTION

In the first preferred embodiment, the glycerin dehydration catalyst according to this invention is prepared by mixing a solution of at least one metal selected from elements belonging to Group 1 to Group 16 of the Periodic Table of Elements or its onium with a solution of heteropolyacid or constituents of heteropolyacid, and of calcinating the resulting solid substance directly or after the resulting solid substance is supported on a carrier.

The unsaturated aldehyde is preferably acrolein and the unsaturated carboxylic acid is preferably acrylic acid.

The solution of at least one metal selected from elements belonging to the Group 1 to Group 16 of the Periodic Table of Elements or onium can be an aqueous solution of halide, hydroxide, carbonate, acetate, nitrate, oxalate, phosphate or sulfate of metal or onium.

The heteropolyacid is known and has several structures such as Keggin type, Dawson type and Anderson type and possess generally such high molecular weight as 700 to 8,500. There are dimer complex forms and those dimer complex are included in the present invention.

The elements belonging to Group 1 to Group 16 of the Periodic Table of Elements may be sodium, potassium, rubidium, cesium, magnesium, calcium, strontium, barium, scandium, yttrium, lanthanide, titanium, zirconium, hafnium, chromium, manganese, rhenium, iron, ruthenium, osmium, cobalt, rhodium, iridium, nickel, palladium, platinum, copper, silver, gold, zinc, gallium, indium, thallium, germanium, tin, lead, bismuth and tellurium. The onium salts of heteropolyacid may be amine salts, ammonium salts, phosphonium salts and sulfonium salts.

Ions of molybdenum and of tungsten form oxoacid in water and the oxoacids polymerize to form the polyoxoacid of high molecular weight. The polymerization may not be effected only with the same kind of oxoacids but also with other kinds of oxoacids. Heteropolyacid is a polyacid possessing polynuclear structure, obtained by condensation of more than two kinds of oxoacids. An atom that forms the center oxoacid is called as “hetero-atom”, while atoms forming oxoacids surrounding the center oxoacid and obtained by the polymerization is called as “poly-atoms”. The heteroatom may be silicon, phosphorus, arsenic, sulfur, iron, cobalt, boron, aluminum, germanium, titanium, zirconium, cerium and chromium. Among them, phosphorus and silicon are preferable. The poly-atoms may be molybdenum, tungsten, vanadium, niobium and tantalum. Among them, molybdenum and tungsten are preferable. The heteropolyacids used in this invention to prepare a glycerin dehydration catalyst may be tungstophosphoric acid, tungstosilicic acid, phosphomolybdic acid and silico molybdic acid. The heteropolyacid may be a mixed coordinate comprising the hetero-atoms of phosphorus or silicon and the poly-atoms are mixed coordinate of molybdenum and tungsten, or mixed coordinate of tungsten and vanadium or mixed coordinate of vanadium and molybdenum.

The constituents of heteropolyacid that can be used in the present invention can be any form that results in the heteropolyacid. The constituents of heteropolyacid may be, for example, a combination of an acid such as phosphoric acid, silicic acid, molybdic acid, tungstic acid, meta tungstic acid and borotungustic acid with a salt such as for example ammonium pertungstate, ammonium phosphate and ammonium metasilicate.

The carrier used in the present invention is not limited specially but the carrier may be silica, diatomaceous earth, alumina, silica alumina, silica magnesia, zirconia, titania, niobia, magnesia, zeolite, silicon carbide, carbide, ceria, boria, ceria-titania, zirconia-ceria, alumina-titanate and alumina-boria. The carrier used in the present invention can be acidic supports listed in Tanabe and al, Studies in Surface Science and Catalysis, Vol 51, 1989, New solid acids and bases, (definition and classification of solid Acids and Bases). Among these carriers, titania, niobia and silica-alumina are preferred. The carrier can be granule and powder and may have any shape such as sphere, pellet, cylindrical body, hollow cylinder body and bar with optional molding aid. The catalyst have preferably a specific surface area of lower than 200 m³/g and more preferably of lower than 100 m³/g. The catalyst can be supported on one of these carriers or on a complex of more than two carriers or on a mixture of these carriers. An amount of the catalyst supported on the carrier can be 5% to 200% by weight, preferably 10 to 150% by weight.

Solvent for preparing the above solution is not limited specially and can be any solvent that can make the solution. Water is preferably used as solvent, so that the solution is preferably an aqueous solution.

In the second preferred embodiment, a catalyst used in a production of acrolein and acrylic acid by dehydration reaction of glycerin according to the present invention, the first mixture can be prepared by one of following methods (1) or (2):
(1) a solution of heteropolyacid or the constituents of heteropolyacid is mixed with a carrier, and a solution of at least one metal selected from elements belonging to Group 1 to Group 16 of the Periodic Table of Elements or its onium is added to the resulting mixture, or
(2) a solution of at least one metal selected from elements belonging to Group 1 to Group 16 of the Periodic Table of Elements or its onium is mixed with a carrier firstly and then a solution of heteropolyacid or constituents of heteropolyacid is added to the resulting mixture.

The mixing can be carried out at ambient temperature (about 20° C.). Higher temperatures of about 40° C. to about 150° C. may be used, if desired. This treatment may be continued, preferably with agitation, for about 0.1 to about 5 hours sufficient to permit the aqueous solution to penetrate the carrier. Suitably, the amount of aqueous solution of at least one metal selected from elements belonging to the Group 1 to Group 16 of the Periodic Table of Elements or onium and the heteropolyacid that is used should be adequate to permit full immersion of the carriers.

At the end of the mixture step, the excess aqueous solution can be evaporated from the treated carriers, or it can be removed from the aqueous solution and permitted to dry in a drying oven.

The resulting solid substance is then calcinated to obtain the catalyst.

From the further aspect, the catalyst used in a production of acrolein and acrylic acid by dehydration reaction of glycerin according to the present invention can be prepared by mixing followings (1) to (3) simultaneously or sequentially to obtain a solid substance:
(1) a solution of heteropolyacid or of constituents of heteropolyacid,
(2) a solution of at least one metal selected from elements belonging to Group 1 to Group 16 of the Periodic Table of Elements or its onium, and
(3) a carrier

The resulting solid substance is then subjected to at least one time of calcination before the solid substance is used in the dehydration reaction of glycerin.

The catalyst according to the present invention used for producing acrolein and acrylic acid from glycerin contains preferably at least one element selected from a group comprising W, Mo and V.

In a preferred embodiment, the alkali metal is preferably cesium and at least a part of protons in the heteropolyacid is exchanged with cesium. It is also possible to exchange at least a part of protons in the heteropolyacid with cesium and a part of remaining protons in the heteropolyacid is exchanged at least partially with at least one cation selected from elements belonging to Group 1 to Group 16 of the Periodic Table of Elements. Acrolein and acrylic acid can be produced at higher yield by using the glycerin dehydration catalyst according to the present invention. Resistance to water is increased by exchanging part of protons contained in the heteropolyacid with cesium, so that the life of catalyst is improved in comparison to heteropolyacid that is inherently water-soluble.

An amount of the aqueous solution of mineral salt of exchanging cation is determined in such a manner that an electric charge of cation to be added is equal to or less than an electric charge of the heteropolyanion. For example, when a cation with charges of 1⁺ is added to a heteropolyanion with charges of 3⁻, the cation is added equal to or less than 3 equivalent to the heteropolyanion, and when a cation with charges of 3⁺ is added to a heteropolyanion with charges of 3⁻, the cation is added equal to or less than 1 equivalent to the heteropolyanion. When a plurality of cations is introduced, an amount of the cation is determined in such a manner that the total electric charge of the cations becomes equal to or less than an electric charge of the heteropolyanion. If an amount of an aqueous solution of inorganic salt or a proportion of the cation(s) to be exchanged with protons become excessive, the activity of catalyst is spoiled or the yields of acrolein and acrylic acid are lowered or the life of catalyst is shortened.

In a variation, the glycerin dehydration catalyst according to this invention contains further at least compound of elements belonging to Group 1 to Group 16 of the Periodic Table of Element in addition to the above compound. The compound of elements belonging to Group 1 to Group 16 of the Periodic Table of Element may be metal salts or onium salts. The metal salt may be salt of tellurium, platinum, palladium, iron, zirconium, copper, cerium, silver and aluminum. The onium salts may be amine salts, ammonium salts, phosphonium salts and sulfonium salts. The metal salt or the onium salt may be prepared from such materials as nitrates, carbonate, sulfates, acetates, hydroxides, oxides and halides of the metals or of onium but are not limited thereto. A proportion of the metal salt is 0.0001 to 60% by weight, preferably 0.001 to 30% by weight in term of the metal salts or the onium salt with respect to the above compound.

The exact nature of bonding of the catalyst composition according to the present invention is not completely understood.

A preferred catalyst for dehydration of glycerin according to the present invention comprises a compound represented by the following general formula (I):

Hₐ A_{b} [X₁Y_{c}Z_{d}Oₑ].nH₂O (I)

in which

H is hydrogen,

A is at least one cation selected from elements belonging to Group 1 to Group 16 of the Periodic Table of Elements except H

X is P or Si,

Y is at least one element selected from the group comprising W [symbol] Mo [symbol] Ti [symbol] Zr [symbol] V [symbol] Nb [symbol] Ta [symbol] Cr [symbol] Mn [symbol] Fe [symbol] Co [symbol] Ni [symbol] Cu [symbol] Zn [symbol] Ga [symbol] In [symbol] Tl [symbol] Sn and Pb,

Z is at least one element selected from the group comprising W [symbol] Mo [symbol] Ti [symbol] Zr [symbol] V [symbol] Nb [symbol] Ta [symbol] Cr [symbol] Mn [symbol] Fe [symbol] Co [symbol] Ni [symbol] Cu [symbol] Zn [symbol] Ga [symbol] In [symbol] Tl [symbol] Sn and Pb, and

a, b, c and satisfying following ranges:

0≦a<9,

0≦b≦9, preferably 0<b≦9

0<c≦12

0≦d<12 and

0<c+d≦12

e is a number determined by the oxidation of the elements and n is any positive number.

In the present invention, the compound represented by the formula (I) is deposited on a carrier or support (“supported catalyst”). In this text, terms of carrier or support have the same meaning.

The carrier used in the present invention is not limited specially but the carrier may be silica, diatomaceous earth, alumina, silica alumina, silica magnesia, zirconia, titania, niobia, magnesia, zeolite, silicon carbide, carbide, ceria, boria, ceria-titania, zirconia-ceria, alumina-titanate and alumina-boria. The carrier can be acidic supports mentioned-above. The catalyst can be supported on one of these carriers or on a complex of more than two carriers or on a mixture of these carriers. An amount of the catalyst supported on the carrier can be 5% to 200% by weight, preferably 10 to 150% by weight.

The resulting supported catalyst can be supported further on at least one another carrier selected from the group comprising silica, diatomaceous earth, alumina, silica alumina, silica magnesia, zirconia, titania, niobia, magnesia, zeolite, silicon carbide, carbide, ceria, boria, ceria-titania, zirconia-ceria, alumina-titanate and alumina-boria. The carrier can be acidic supports mentioned-above.

An amount of the above-mentioned loaded compound supported on the carrier is 5 to 99.9% by weight, preferably 5 to 90% by weight to the weight of the carrier.

The carrier can be granule and powder and may have any shape such as sphere, pellet, cylindrical body, hollow cylinder body and bar with optional molding aid.

The catalyst may have any shape and can be granule, powder or monolith. In case of gas phase reactions, however, it is preferable to mold the catalyst into a shape of monolith, sphere, pellets, cylinder, hollow cylinder, bar or the like optionally with adding a molding aid or the catalyst is shaped into these configurations together with carrier and optional auxiliary agents. A size of molded catalyst is for example 1 to 10 mm for a fixed bed and less than 1 mm for a fluidized bed.

In case of a fluidized bed reactor for the process for preparing acrolein, it is preferred to have a powder with appropriate average particle size distribution namely between 40 and 300 μm, preferably between 60 and 150 μm.

In this text, wordings of “firing” or “calcination” are used in the same meaning. Namely, the catalyst composition according to the present invention can be prepared by the step of the above mixing and then of drying and firing the resulting solid mixture obtained. In a variation, the resulting solid mixture may be impregnated further with a solution of other elements used for improving durability or for activity before calcination.

The catalyst according to the present invention used in the glycerin dehydration may be anhydrides or hydrates. In fact, they can be used after pretreatment of firing and vacuum drying or without pretreatment.

The calcination can be carried out in air or under inert gas such as nitrogen, helium and argon or under an atmosphere of mixed gas of air and inert gas usually or under reduction gas such as hydrogen or an atmosphere of mixed gas of hydrogen and inert gas in a furnace such as muffle furnace, rotary kiln, fluidized bed furnace. The furnace is not limited specially. The calcination can be effected even in a reaction tube that is used for the glycerin dehydration reaction. The firing temperature is usually 150 to 900° C., preferably 200 to 800° C. and more preferably 350 to 650° C. This can be determined by routine experimentation for a particular catalyst. Temperatures above 900° C. should be avoided. The calcination is continued usually for 0.5 to 20 hours.

The dehydration reaction of glycerin according to this invention can be carried out in gas phase or in liquid phase and the gas phase is preferable. The gas phase reaction can be carried out in a variety of reactors such as fixed bed, fluidized bed, circulating fluidized bed and moving bed. Among them, the fixed bed or the fluidized bed is preferable. Regeneration of the catalyst can be effected outside the reactor. When the catalyst is taken out of a reactor system for regeneration, the catalyst is burnt in air or in oxygen-containing gas. In case of liquid phase reaction, usual general reactors for liquid reactions for solid catalysts can be used. Since the difference in boiling point between glycerin (290° C.) and acrolein and acrylic acid is big, the reaction is effected preferably at relatively lower temperatures so as to distil out acrolein continuously.

The reaction temperature for producing acrolein and acrylic acid by dehydration of glycerin in gas phase is effected preferably at a temperature of 200° C. to 450° C. If the temperature is lower than 200° C., the life of catalyst will be shortened due to polymerization and carbonization of glycerin and of reaction products because the boiling point of glycerin is high. On the contrary, if the temperature exceeds 450° C., the selectivity of acrolein and acrylic acid will be lowered due to increment in parallel reactions and successive reactions. Therefore, more preferable reaction temperature is 250° C. to 350° C.

The reaction for producing acrolein and acrylic acid by dehydration of glycerin in gas phase is effected pressurized conditions of 0.01 MPa to 1 MPa. Under higher pressures than 1 MPa, gasified glycerin will be re-liquefied and deposition of carbon will be promoted by higher pressure so that the life of catalyst will be shortened.

A feed rate of a material gas is preferably 500 to 10,000 h⁻¹ in term of the space velocity of GHSV. The selectivity will be lowered if the GHSV becomes lower than 500 h⁻¹ due to successive reactions. On the contrary, if the GHSV exceeds 10,000 h⁻¹, the conversion will be lowered.

The reaction temperature of the liquid phase reaction is preferably from 150° C. to 350° C. The selectivity will be spoiled under lower temperatures although the conversion is improved. The reaction can be carried under a pressurized condition of 0.01 MPa to 7 MPa.

The material of glycerin is easily available in a form of aqueous solution of glycerin. Concentration of the aqueous solution of glycerin is from 5% to 90% by weight and more preferably 10% to 50% by weight. Too high concentration of glycerin will result in such problems as production of glycerin ethers or undesirable reaction between the resulting acrolein and acrylic acid and material glycerin. Temperature that is necessary to gasify glycerin is increased.

### EXAMPLES

Now, the present invention will be explained in much detail with referring several examples, but this invention should not be limited to those described in following examples. In the following Examples and Comparative Examples, % means mole %.

Several catalysts of cesium salt of tungstophosphoric acid(CsPW) supported on a variety of carriers were prepared as following.

### Example 1

### CsPW/TiO₂

Pellet of TiO₂ (ST31119, product of Saint Gobain) was ground and passed through a sieve to obtain TiO₂ powder of 300 to 500 μm, which was then dried for one night at 110° C. 10 g of tungstophosphoric acid (H₃[PW₁₂O₄₀]nH₂O, n=about 30, a product of Nippon Inorganic Colour & Chemical Co., Ltd.) (PW) was dissolved in 150 ml of pure water to obtain an aqueous solution of tungstophosphoric acid. This aqueous solution of tungstophosphoric acid was added to 19.65 g of the TiO2 powder and stirred for 2 hours at ambient temperature to obtain a slurry of PW/TiO₂.

In another beaker, 2.26 g of 48.5 wt % of cesium hydroxide (CsOH) was dissolved in 10 ml of water to obtain an aqueous solution of cesium hydroxide. This aqueous solution of cesium hydroxide was added drop wise to the white slurry of PW/TiO₂ by using a dropping funnel under stiffing. The resulting white slurry was dried in a rotary evaporator at 60° C. under reduced pressure and then was further dried at 120° C. in a drier under ambient pressure for 10 hours. The resulting white powder was fired in a muffle furnace at 500° C. in air for 3 hours to obtain CsPW supported titania catalyst (CsPW(30 wt %)/TiO₂).

Then, the catalyst was evaluated in a fixed bed reactor operated under pressure by passing material flow through the fixed bed. The resulting catalyst powder was compacted and then crushed. Crushed particles were sieved to obtain particles of 9 to 12 mech. 10 cc of the catalyst granules or particles was packed in a SUS reaction tube (diameter of 20 mm). An aqueous solution of glycerin (concentration of 30% by weight) was fed to an evaporator at a flow rate of 21 g/hr by a pump so that glycerin was gasified at 300° C. The resulting gasified glycerin was passed through the fixed catalyst bed together with air. The fixed catalyst bed was heated at a temperature of 260° C. to 350° C. Feed gas had following composition in mol %: glycerin:oxygen:nitrogen:water=6.3:4.0:14.9:74.8. GHSV was 2445 h⁻¹. An internal pressure of the reactor was adjusted to a relative pressure of 0.2 MPa.

Products were condensed in a condenser and quantitative-analyzed by a gas chromatograph (GC-7890A, a product of Agilent, DB-WAX etr column). Proportions of products were corrected in factors from the results of the gas chromatograph to determine absolute amounts of products to calculate the conversion (%) of material (the conversion of glycerin), selectivity of products (the selectivity of acrolein etc.) and the yield of objective substance (the yield of acrolein) from an amount of glycerin fed, an amount of glycerin remained and amounts of the products by following equations:

The conversion (%) of material=(a mole number of material reacted/a mole number of material supplied)×100

The selectivity (%) of objective substance=(a mole number of products obtained/a mole number of material reacted)×100

The yield (%) of products=(a mole number of products obtained/a mole number of material fed)×100

Results are summarized in Table 1.

### Example 2

### CsPW/Nb₂O₅

Powder of Nb₂O₅ (product of Mitsui Mining & Smelting Co., Ltd.) was dried at 110° C. for one night. 30 g of tungstophosphoric acid (H₃[PW₁₂O₄₀] nH₂O (n=about 30, a product of Nippon Inorganic Colour & Chemical Co., Ltd.) was dissolved in 450 ml of pure water to obtain an aqueous solution of tungstophosphoric acid. This aqueous solution of tungstophosphoric acid was added to 58.94 g of the Nb₂O₅ powder and stirred for 2 hours at ambient temperature. The resulting slurry was dried in a rotary evaporator at 60° C. and then was further dried at 120° C. in a drier under ambient pressure at 120° C. for 10 hours. The resulting powder was fired in a muffle furnace at 250° C. in air for 3 hours to obtain PW/Nb₂O₅ powder. 30 g of the PW/Nb₂O₅ powder was added to 85 ml of water under stirring. In another beaker, 2.41 g of 48.5 wt % cesium hydroxide (CsOH) was dissolved in 10 ml of water to obtain an aqueous solution of cesium hydroxide. This aqueous solution of cesium hydroxide was added drop wise under stiffing to the white slurry of PW/Nb₂O₅. The resulting white slurry was dried in a rotary evaporator at 60° C. under reduced pressure and then was further dried at 120° C. in a drier under ambient pressure at 120° C. for 10 hours. The resulting white powder was fired in a muffle furnace at 500° C. in air for 3 hours to obtain CsPW supporting niobia catalyst (CsPW(30 wt %)/Nb₂O₅).

The resulting catalyst was evaluated by the same method as Example 1 under tha same conditions.

### Example 3

### CsPW/SiO₂—Al₂O₃

300 g of cesium salt of tungstophosphoric acid (Cs_{2.5} H_{0.5}[PW₁₂O₄₀], a product of Nippon Inorganic Colour & Chemical Co., Ltd.) (CsPW) was mixed with 15 g of SiO₂—Al₂O₃ powder used as a molding additive. 300 g of spherical silica-alumina support having an average particle size of 3.8 mm was put into a rolling granulating machine. Onto the spherical silica-alumina support, the mixture of CsPW was added to obtain a spherical supported catalyst in which the CsPW was supported on the spherical silica-alumina support at a support ratio of 50% by weight. The resulting catalyst was dried at 150° C. for 6 hours under ambient pressure and then fired in air at 500° C. for 3 hours to obtain a spherical CsPW(50 wt %)/SiO₂—Al₂O₃ catalyst in which CsPW was supported on SiO₂—Al₂O₃ spherical carrier at a coverage ratio of 50 wt %.

Then, the reactivity of the catalyst was evaluated in a fixed bed reactor operated under pressure. 30 cc of the spherical catalyst was packed in a SUS reaction tube (diameter of 20 mm). An aqueous solution of glycerin (concentration of 30% by weight) was fed to an evaporator at a flow rate of 63 g/hr by a pump so that glycerin was gasified at 300° C. The resulting gasified glycerin was passed through the fixed catalyst bed together with air. The fixed catalyst bed was heated at a temperature of 260° C. to 350° C. Feed gas had following composition in mol %: glycerin:oxygen:nitrogen:water=6.3:4.0:14.9:74.8. GHSV was 2445 h⁻¹. An internal pressure of the reactor was adjusted to a relative pressure of 0.2 MPa.

Recovery, quantitative analysis and calculation of products were effected by the same method as Example 1.

### Example 4

### CsPW/TiO₂/SaO₂—Al₂O₃

Pellet of TiO₂ (ST31119, product of Saint Gobain) was ground and passed through a sieve to obtain TiO₂ powder of 300 to 500 μm, which was then dried for one night at 110° C. 350 g of tungstophosphoric acid (H₃[PW₁₂O₄₀] nH₂O, n=about 30, a product of Nippon Inorganic Colour & Chemical Co., Ltd.) (PW) was dissolved in 1900 ml of pure water to obtain an aqueous solution of PW. This aqueous solution of PW was added to 442 g of the TiO₂ powder and stirred for 2 hours at ambient temperature to obtain a slurry of PW/TiO₂.

In another beaker, 79.06 g of 48.5 wt % of cesium hydroxide (CsOH) was dissolved in 25 ml of water to obtain an aqueous solution of cesium hydroxide. This aqueous solution of cesium hydroxide was added drop wise to the white slurry of PW/TiO₂ by using a dropping funnel under stiffing. The resulting white slurry was dried in a rotary evaporator at 60° C. under reduced pressure and then was further dried at 120° C. in a drier under ambient pressure for 10 hours to obtain CsPW suppored titania powder (CsPW(40 wt %)/TiO₂).

300 g of spherical silica alumina support having an average particle size of 3.8 mm was put into a rolling granulating machine. Onto the spherical silica-alumina support, the CsPW supported titania powder (CsPW(40 wt %)/TiO₂) was added to obtain a spherical supported catalyst in which the (CsPW(40 wt %)/TiO₂) was supported on the support at a support ratio of 50% by weight. The resulting catalyst was dried at 150° C. for 6 hours under ambient pressure and then fired in air at 500° C. for 3 hours to obtain a spherical CsPW(40 wt %)/TiO₂/SiO₂—Al₂O₃ catalyst in which CsPW(40 wt %)/TiO₂ was supported on SiO₂—Al₂O₃ spherical carrier at a coverage ratio of 50 wt %.

The resulting catalyst was evaluated by the same method as Example 3.

As comparison, non-supported cesium salt of tungstophosphoric acid (product of Nippon Inorganic Colour & Chemical Co., Ltd.) (CsPW) was used. Namely, a compound in which protons in a heteropolyacid are exchanged at least partially with at least one cation selected from elements belonging to Group 1 to Group 16 of the Periodic Table of Elements was used without carrier.

### Comparative Example 1

### CsPW

CsPW powder was fired in a Muffle furnace at 500° C. in air for 3 hours to obtain a CsPW catalyst. The resulting catalyst was evaluated in the same fixed bed as Example 1 but the reaction was effected under ambient pressure.

### Comparative Example 2

### CsPW

CsPW powder was fired in a Muffle furnace at 500° C. in air for 3 hours to obtain a CsPW catalyst. The resulting catalyst was evaluated by the same conditions as Example 1.

TABLE 1

| | Example | | | | | |
|---|---|---|---|---|---|---|
| | 1 | 2 | 3 | 4 | Comparative | |
| | CsPW/ | CsPW/ | CsPW/ | CsPW/TiO₂/ | 1 | 2 |
| Catalyst | TiO₂ | Nb₂O₅ | SiO₂—Al₂O₃ | SiO₂—Al₂O₃ | CsPW | CsPW |
| Pressure (MPa) | 0.2 | 0.2 | 0.2 | 0.2 | Atmosphere | 0.2 |
| Reaction temperature (° C.) | 280 | 280 | 280 | 300 | 300 | 300 |
| Glycerin conversion (%) | 99.8 | 99.3 | 94.8 | 100 | 100 | 100 |
| Acrolein yield (%) | 67 | 75 | 77 | 76 | 82 | 50 |
| Acrolein selectivity (%) | 67 | 75 | 82 | 76 | 82 | 50 |
| Hydroxypropanone yield (%) | 0.0 | 0.2 | 0.4 | 0.0 | 0.0 | 0.1 |
| Acetaldehyde yield (%) | 6.1 | 2.5 | 3.0 | 6.2 | 1.8 | 3.2 |
| Propanaldehyde yield (%) | 0.4 | 0.5 | 0.2 | 0.6 | 0.6 | 0.5 |
| Acrylic acid yield (%) | 4.6 | 2.1 | 1.6 | 1.8 | 3.0 | 9.3 |
| CO yield (%) | 6.8 | 5.4 | 2.4 | 5.3 | 2.5 | 10.1 |
| CO₂ yield (%) | 5.6 | 4.0 | 1.9 | 4.5 | 1.8 | 7.2 |

### Example 5

### CsPW/TiO2 in a Fluidized Bed Reactor

Pellets of TiO2 (ST31119, product of Saint Gobain) were ground and passed through a sieve to obtain TiO2 powder of 50 to 100 μm, which was then dried for one night at 110° C. 350 g of tungstophosphoric acid (H3[PW12O40] nH2O, n=about 30, a product of Nippon Inorganic Colour & Chemical Co., Ltd.) (PW) was dissolved in 1900 ml of pure water to obtain an aqueous solution of tungstophosphoric acid. This aqueous solution of tungstophosphoric acid was added to 291 g of the TiO₂ powder and stirred for 2 hours at ambient temperature to obtain a slurry of PW/TiO2.

In another beaker, 79.06 g of 48.5 wt % of cesium hydroxide (CsOH) was dissolved in 25 ml of water to obtain an aqueous solution of cesium hydroxide. This aqueous solution of cesium hydroxide was added dropwise to the white slurry of PW/TiO2 by using a dropping funnel under stirring. The resulting white slurry was dried in a rotary evaporator at 60° C. under reduced pressure and then was further dried at 120° C. in a drier under ambient pressure for 10 hours. The resulting white powder was fired in a muffle furnace at 500° C. in air for 3 hours to obtain CsPW supported titania catalyst (CsPW(50 wt %)/TiO2). The resulting catalyst powder was ground and passed through a sieve to obtain TiO2 powder of 50 to 100 μm.

Then, the catalyst was evaluated in a fluidized bed reactor. Thus, 142 ml of the catalytical powder was charged in a stainless steel reaction tube (diameter of 50 mm). An aqueous solution of glycerin (concentration of 50% by weight) at a flow rate of 136 g/hr and a flow of 170 normal l/hr of nitrogen and 10 normal l/hr of oxygen were fed to an evaporator heated at 280° C. The resulting gaseous flow was fed at the bottom of the reaction tube through a 2 μm grid. The fluidized bed reactor tube was heated at 280° C. Feed gas had following composition in mol %: glycerin:oxygen:nitrogen:water=5.9:3.6:60.4:30.1. GHSV was 1980 h⁻¹. Internal pressure of the reactor was adjusted to a relative pressure of 0.01 MPa. The gaseous outlet of the reactor was passed to a cyclone and sent to a cooled condensation column in which cold water is injected at the top. Products were quantitatively analyzed by gas chromatography (for liquid phase: HP 6890 Agilent, FFAP column, FID detector; for gas phase: CP4900 Varian, Silicaplot and Molecular Sieve 5A, TCD detectors).

Results are summarized in table 2.

TABLE 2

| | Example 5 |
|---|---|
| Catalyst | CsPW/TiO₂ |
| Pressure (MPa) | 0.01 |
| Reactor temperature (° C.) | 280 |
| Glycerin conversion (%) | 99 |
| Acrolein yield (%) | 58 |
| Acrolein selectivity (%) | 58 |
| Hydroxypropanone yield (%) | 0.7 |
| Acetaldehyde (%) | 2.8 |
| Propanaldehyde yield (%) | 0.7 |
| Acrylic acid yield (%) | 0.6 |
| CO yield (%) | 3.8 |
| CO₂ yield (%) | 2.5 |

From the comparison between Examples and Comparative Examples, followings are observed:
(1) The highest yield of acrolein can be such high as 77% which is not so different from a case operated under atmospheric pressure owing to high performance of the catalyst according to the present invention. In fact, the comparative catalyst comprising a compound whose protons in heteropoly acid such as PW and SiW are exchanged with alkali metal like Cs is supported on a carrier such as TiO₂, Nb₂O₅ and SiO₂—Al₂O₃ can be used even under such a severe condition as under pressurized condition when acrolein and acrylic acid was produced by dehydration reaction of glycerin.
(2) In Comparative Examples, the catalyst comprising a compound whose protons in heteropoly acid such as PW and SiW are exchanged with alkali metal like Cs is excessively oxidized under a pressurized condition so that the acrolein yield is lowered greatly down to 50%, although the acrolein yield is such high as 82% when the reaction is effected at atmospheric pressure.
(3) The supported catalyst according to the present invention which is loaded at several times higher load comparing to a non-supported catalyst in which only cation is exchanged shows nearly equal acrolein yield under the pressurized condition.

1. A process for preparing a catalyst used in a production of acrolein and acrylic acid by dehydration reaction of glycerin, characterized by the steps of mixing a solution of at least one metal selected from elements belonging to Group 1 to Group 16 of the Periodic Table of Elements or its onium with a solution of heteropolyacid or constituents of heteropolyacid, and of calcinating the resulting solid substance directly or after the resulting solid substance is supported on a carrier.

2. A process for preparing a catalyst used in a production of acrolein and acrylic acid by dehydration reaction of glycerin, characterized by the steps of either mixing a solution of heteropolyacid or constituents of heteropolyacid with a carrier, and then adding a solution of at least one metal selected from elements belonging to Group 1 to Group 16 of the Periodic Table of Elements or its onium to the resulting mixture, or mixing a solution of at least one metal selected from elements belonging to Group 1 to Group 16 of the Periodic Table of Elements or its onium with a carrier, and then adding a solution of heteropolyacid or constituents of heteropolyacid to the resulting mixture, and then calcinating the resulting solid substance to obtain the catalyst.

3. A process for preparing a catalyst used in a production of acrolein and acrylic acid by dehydration reaction of glycerin, characterized by mixing a solution of heteropolyacid or constituents of heteropolyacid, a solution of at least one metal selected from elements belonging to Group 1 to Group 16 of the Periodic Table of Elements or its onium and a carrier to obtain a solid substance, and then effecting at least one time of calcination before said solid substance is used in the dehydration reaction of glycerin.

4. The process claim 1, in which the calcination is carried out in air, in inert gas or in a mixture of oxygen and inert gas or under a reduced gas of hydrogen and inert gas.

5. The process of claim 1, in which the calcination is effected at a temperature of 150° C. to 900° C. for 0.5 to 20 hours.

6. A catalyst used in a production of acrolein and acrylic acid by dehydration reaction of glycerin, obtained by the process according to claim 1.

7. The catalyst of claim 6, comprising a compound represented by the formula (I):
HₐA_{b}[X₁Y_{c}Z_{d}Oₑ].nH₂O (I)
in which
H is hydrogen,
A is more than one cation selected from elements belonging to Group 1 to Group 16 of the Periodic Table of Elements except hydrogen,
X is P or Si,
Y is more than one element selected from the group comprising W [symbol] Mo [symbol] Ti [symbol] Zr [symbol] V [symbol] Nb [symbol] Ta [symbol] Cr [symbol] Mn [symbol] Fe [symbol] Co [symbol] Ni [symbol] Cu [symbol] Zn [symbol] Ga [symbol] In [symbol] Tl [symbol] Sn and Pb,
Z is more than one element selected from the group comprising W [symbol] Mo [symbol] Ti [symbol] Zr [symbol] V [symbol] Nb [symbol] Ta [symbol] Cr [symbol] Mn [symbol] Fe [symbol] Co [symbol] Ni [symbol] Cu [symbol] Zn [symbol] Ga [symbol] In [symbol] Tl [symbol] Sn and Pb,
a, b, c, d and n satisfying following ranges:
0≦a<9
0≦b≦9, preferably 0<b≦9
0<c≦12
0≦d<12 and 0<c+d≦12
n≧0
and e is a number determined by the oxidation of the elements.
and the compound represented by the formula (I) being deposited on a carrier

8. A process for preparing acrolein by catalytic dehydration of glycerin under a pressurized condition and carried out in the presence of a catalyst prepared according to claim 1.

9. The process of claim 8 in which the dehydration of glycerin is effected in the presence of oxygen gas.

10. The process of claim 8 in which the dehydration of glycerin is effected in the presence of a gas containing propylene.

11. The process of claim 8 carried out in a plate heat exchanger type reactor or in a fixed bed reactor or in a fluidized bed type reactor or in a circulating fluidized bed or in a moving bed.

12. The process of claim 8, wherein the catalytic dehydration of glycerin is effected under a pressurized condition of relative pressure of 0.01 MPa to 1 MPa.

13. The process of claim 8, wherein the resulting acrolein is further oxidized to produce acrylic acid.

14. The process of claim 13 having an intermediate step of partial condensation and removal of water and heavy by-products issuing from the dehydration step.

15. A process for preparing acrylonitrile, characterized in that acrolein obtained by the process of claim 8 is subjected to ammoxidation.

16. A process for preparing acrylic acid comprising a first step of catalytic dehydration of glycerin by the process of claim 8 and a second step of gas phase oxidation of the gaseous reaction product containing acrolein formed by the dehydration reaction, then collecting the resultant acrylic acid as a solution by using water or a solvent and purifying the resultant solution containing acrylic acid by using for example distillation and/or crystallization.
